(19) United States
(12) Patent Application Publication
(10) Pub. No.: US 2014/0005530 A1
(43) Pub. Date: Jan. 2, 2014

(54) ULTRASOUND IMAGING METHOD AND ULTRASOUND IMAGING APPARATUS

(71) Applicant: General Electric Company, Schenectady, NY (US)

(72) Inventors: Gang LIU, Shen Yang (CN); Yiming ZHAO, Zhou Shan (CN); Wenting XIA, Wu Xi (CN); Lawrence Guy Ten Eyck, Elliicott City, MD (US)

(21) Appl. No.: 13/928,404

(22) Filed: Jun. 27, 2013

(30) Foreign Application Priority Data
Jun. 29, 2012 (CN) 201210220551.X

(51) Int. Cl. A61B 8/08; A61B 8/00; A61M 25/01; A61B 8/14

(52) U.S. Cl. CPC A61B 8/0891; A61B 8/14; A61B 8/0883; A61B 8/54; A61B 8/0841; A61M 25/0108
USPC 600/424; 600/443

(57) ABSTRACT

An ultrasound imaging apparatus is provided. The apparatus comprises an ultrasound probe configured to perform ultrasound sweeping of a region of interest including a target location and a target vessel in a real time volume ultrasound scan mode, an image acquiring unit configured to acquire three-dimension scan data of the target location and the target vessel, and a controlling unit configured to calculate the target vessel centerline length based on the acquired three-dimension scan data. The apparatus further comprises an image processing unit configured to automatically mark distance scales from the critical line located at the target location along the target vessel centerline at predetermined intervals based on the calculated centerline length, and a displaying unit configured to displaying the target location and the target vessel centerline marked with distance scales in real time.

### TECHNICAL FIELD

Embodiments of the present invention relate to the field of ultrasound imaging, and in particular to an ultrasound imaging method and an ultrasound imaging apparatus for guiding the placement of a catheter in a vessel in real time by distance scales marked along the vessel centerline.

### BACKGROUND

Some interventional surgical procedures need to insert medical instruments, such as a catheter, into a blood vessel of a patient. As shown in FIG. 2, the catheter is inserted along the blood vessel and finally positioned at a target location, such as the heart entrance of the patient, for subsequent medical processes. During the insertion of the catheter along the blood vessel, such as during a PICC (Peripherally Inserted Central Catheter) procedure, the distance between the catheter tip and the target location, such as the heart entrance, is very important to place the catheter in right position.

Presently, a surgeon typically determines the position of the catheter tip in the blood vessel indirectly through catheter inserting length combined with catheter body's scale. This method cannot show the exact distance between catheter tip and target location because the catheter may be curved in the blood vessel. Presently, the position of the catheter should thus be determined by additionally using an x-ray imaging technology after the insertion of the catheter is done. X-ray imaging may achieve a resolution that is sufficient for a surgeon to differentiate micro vessels, but x-ray imaging may bring about complications related to radiation.

Presently, for debilitated patients such as infants, a real time three-dimension ultrasound imaging technology is used instead of x-ray imaging technology to help surgeons in performing catheter inserting procedures. The ultrasound imaging technology uses ultrasound waves as information carrier, and images the structure inside a body, and there is a correspondence relationship between the picture information thereof and actual structure of a body in spatial and temporal distribution. The medical ultrasound imaging technology uses ultrasound, which will generate echoes with different acoustic intensities due to different acoustic characteristic impedances of different tissues and organs encountered when it travels in a body, to establish a picture. Since the ultrasound imaging technology has advantages of being safe, reliable, real time and no radiation, it is used by more and more surgeons in guiding interventional surgical procedures such as catheter insertion.

Although, to a certain degree, the ultrasound imaging technology can help surgeons in determining the insertion depth of the catheter, since blood vessels in a body are of space curves and blood vessels vary with physiological events such as heart beats, existing ultrasound imaging technologies are hard to exactly determine the distance between the catheter tip and the target location such as the heart entrance, and hence are hard to take a quantitative measurement of the distance.

Therefore, there is a need to provide a novel ultrasound imaging method and apparatus for quantitatively measuring the exact distance between a catheter tip and a target location and dynamically determining the exact position of the catheter tip in real time.

### SUMMARY OF THE INVENTION

Embodiments of the present invention provide an ultrasound imaging method and an ultrasound imaging apparatus that can solve the above-mentioned problems.

In an embodiment of the present invention, there is provided an ultrasound imaging method. The method comprises steps of acquiring three-dimension scan data of a target location and a target vessel by using a real time volume ultrasound scan mode, and calculating the centerline length of the target vessel based on the acquired three-dimension scan data. Additionally, the method comprises automatically marking distance scales from the critical line located at the target location along the target centerline at predetermined intervals based on the calculated centerline length, and displaying the target location and the target vessel centerline marked with distance scales in real time.

In the ultrasound imaging method of an embodiment of the present invention, the step of displaying the target location and the target vessel centerline marked with distance scales in real time comprises projecting the three-dimension scan data marked with distance scales onto a two-dimension coordinate plane, and displaying the projected two-dimension scan data in real time.

In the ultrasound imaging method of an embodiment of the present invention, when three-dimension scan data are acquired, the swept direction of the ultrasound probe is substantially perpendicular to the longitudinal axis of the target vessel.

In the ultrasound imaging method of an embodiment of the present invention, the marked distance scales have the same or different intervals.

In the ultrasound imaging method of an embodiment of the present invention, the target location is a heart, and the critical line is located at the heart entrance.

In the ultrasound imaging method of an embodiment of the present invention, the step of calculating the centerline length of the target vessel based on the acquired three-dimension scan data comprises creating a target vessel mathematic model in the three-dimension scan data, detecting and tracking the vessel centerline in real time based on the three-dimension scan data by using the created mathematic model, and automatically calculating the target vessel centerline length based on the detected and tracked target vessel centerline.

In the ultrasound imaging method of an embodiment of the present invention, the acquired three-dimension scan data include a plurality of two-dimension vessel frames along the longitudinal axis of the target vessel.

In the ultrasound imaging method of an embodiment of the present invention, the step of detecting and tracking the target vessel centerline in real time based on the three-dimension scan data by using the created mathematic model comprises detecting and tracking the target vessel centerline by applying a real time image segmentation algorithm.

In the ultrasound imaging method of an embodiment of the present invention, the step of detecting and tracking the target vessel centerline by applying a real time image segmentation algorithm comprises detecting and tracking the target vessel centerline in real time on a frame-by-frame basis by using a Kalman filter in order to obtain the target vessel centerline coordinate of each two-dimension vessel frame in a three-dimension ultrasound beam space.

In the ultrasound imaging method of an embodiment of the present invention, the step of automatically calculating the target vessel centerline length based on the detected and tracked target vessel centerline comprises transforming target vessel centerline coordinates from three-dimension ultrasound beam space to Cartesian space, and calculating the target vessel centerline length by using target vessel centerline coordinates in Cartesian space.

In the ultrasound imaging method of an embodiment of the present invention, the step of transforming target vessel centerline coordinates from three-dimension ultrasound beam space to Cartesian space comprises transforming target vessel centerline coordinates from three-dimension ultrasound beam space to three-dimension acquisition coordinate system, and transforming target vessel centerline coordinates from three-dimension acquisition coordinate system to Cartesian coordinate system.

In an embodiment of the present invention, there is provided an ultrasound imaging apparatus. The apparatus comprises an ultrasound probe configured to perform ultrasound sweeping of a region of interest including the target location and the target vessel in a real time volume ultrasound scan mode, an image acquiring unit configured to acquire three-dimension scan data of the target location and the target vessel, and a controlling unit configured to calculate the target vessel centerline length based on the acquired three-dimension scan data. Additionally, the apparatus comprises an image processing unit configured to automatically mark distance scales from the critical line located at the target location along the target vessel centerline at predetermined intervals based on the calculated centerline length, and a displaying unit configured to displaying the target location and the target vessel centerline marked with distance scales in real time.

In the ultrasound imaging of an embodiment of the present invention, the image processing unit is further configured to project the three-dimension scan data marked with distance scale onto a two-dimension coordinate plane, and the displaying unit is further configured to display the projected two-dimension scan data in real time.

In the ultrasound imaging apparatus of an embodiment of the present invention, the swept direction of the ultrasound probe is substantially perpendicular to the longitudinal axis of the target vessel.

In the ultrasound imaging apparatus of an embodiment of the present invention, the marked distance scales have the same or different intervals.

In the ultrasound imaging apparatus of an embodiment of the present invention, the target location is a heart, and the critical line is located at the heart entrance.

In the ultrasound imaging apparatus of an embodiment of the present invention, when the catheter is inserted along the target vessel centerline, the image acquiring unit is further configured to acquire the three-dimension scan data of the catheter tip, and the displaying unit is further configured to display the catheter tip together with the target location and the target vessel centerline.

The ultrasound imaging apparatus of an embodiment of the present invention also comprises a detecting unit and an alarm. The detecting unit is used to determine the position of a catheter tip of the catheter having a position sensor disposed at its tip when it is inserted along the target vessel. The alarm is used to issue an alarm when the catheter tip provided with a position sensor has reached a predetermined distance from the critical line along the target vessel centerline. In an embodiment, the alarm may be audible and/or visual such as, for example, a speaker or an indicator.

In the ultrasound imaging apparatus of an embodiment of the present invention, the controlling unit comprises a mathematic model creating module for creating a mathematic model of the target vessel in three-dimension scan data, a detecting and tracking module for detecting and tracking the target vessel centerline in real time based on three-dimension scan data by using the created mathematic model, and a calculating module for automatically calculating the target vessel centerline length based on the detected and tracked target vessel centerline.

In the ultrasound imaging apparatus of an embodiment of the present invention, the three-dimension scan data acquired by the image acquiring unit comprises a plurality of two-dimension vessel frames along the longitudinal axis of the target vessel.

In the ultrasound imaging apparatus of an embodiment of the present invention, the detecting and tracking module is configured to detect and track the target vessel centerline by applying a real time image segmentation algorithm.

In the ultrasound imaging apparatus of an embodiment of the present invention, the detecting and tracking module also comprises a Kalman filter for detecting and tracking the target vessel centerline in real time on a frame-by-frame basis in order to obtain the target vessel centerline coordinates of each two-dimension vessel frame in three-dimension ultrasound beam space.

In the ultrasound imaging apparatus of an embodiment of the present invention, the calculating module comprises a coordinate transforming unit for transforming the target vessel centerline coordinates from three-dimension ultrasound beam space to Cartesian space, and a calculating unit for calculating the target vessel centerline length based on the target vessel centerline coordinates in Cartesian space.

In the ultrasound imaging apparatus of an embodiment of the present invention, the coordinate transforming unit transforms the target vessel centerline coordinates from a three-dimension ultrasound beam space to a three-dimension acquisition coordinate system, and transforms the target vessel centerline coordinates from a three-dimension acquisition coordinate system to a Cartesian coordinate system.

Using the ultrasound imaging method and the ultrasound imaging apparatus of an embodiment of the present invention, it is possible to guide the real time placement of a catheter tip by marking distance scales along the target vessel centerline, and to guide the catheter insertion route through the real time varying vessel centerline when the target vessel is changed dynamically due to physiological events such as heart beats. Since the present invention is based on ultrasound scan instead of x-ray scan, and since ultrasound imaging does not involve harmful radiation, it is safely and reliably used for various patients including debilitated patients such as elderly patients and newborn babies. Furthermore, the present invention can support various catheters, such as ordinary catheters, and when a catheter tip is provided with a position sensor, the present invention may issue an alarm automatically when the catheter tip is proximate to or at the predetermined critical line of the heart entrance.

### BRIEF DESCRIPTION OF DRAWINGS

Hereinafter, some embodiments of the present invention will be described in details with reference to the accompanying drawings. The same reference numbers are used to denote the same or similar parts throughout these figures, in which:

FIG. 1 is a general structural diagram illustrating an ultrasound imaging apparatus according to an embodiment of the present invention;

FIG. 2 is a schematic diagram illustrating catheter insertion along a blood vessel;

FIG. 3 illustrates a real time volume ultrasound scan mode of an ultrasound probe;

FIG. 4 is a structural diagram illustrating a controlling unit;

FIG. 5 illustrates an mathematic model of a blood vessel;

FIG. 6 is a schematic diagram illustrating real time detection and track of a vessel centerline;

FIG. 7 is a schematic diagram illustrating the transformation of vessel centerline coordinates from beam space to Cartesian space;

FIG. 8 illustrates a process of calculating a vessel centerline length;

FIG. 9 is a schematic diagram illustrating the real time marking of distance scales from a critical line along a vessel centerline;

FIG. 10 is a flowchart of an ultrasound imaging method according to an embodiment of the present invention;

FIG. 11 is a flowchart of a method for calculating a vessel centerline length based on the acquired three-dimension ultrasound scan data; and

FIG. 12 is a flowchart of a method for automatically calculating its length based on the detected and tracked vessel centerline.

### DETAILED DESCRIPTION

In the following detailed description, some embodiments of the present invention will be described with reference to the accompanying drawings. Those skilled in the art will appreciate that the present invention should not be considered as limited to these embodiments.

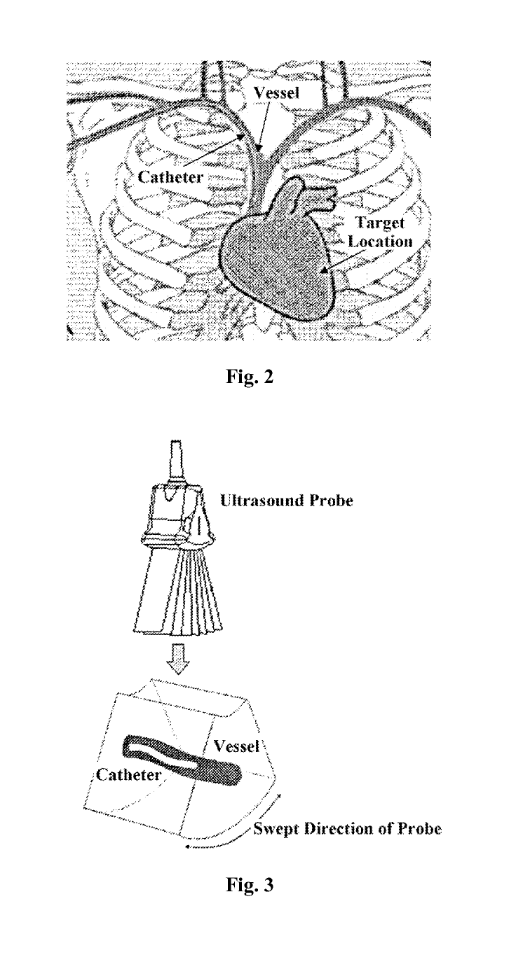

FIG. 2 is a schematic diagram illustrating a catheter inserting procedure performed by a surgeon along a blood vessel. As shown in FIG. 2, the catheter is inserted along a blood vessel until the catheter tip is positioned at the target location (e.g., heart entrance). As described above, during the catheter insertion, the distance between the catheter tip and the target location is important to place the catheter in the right position. With the use of the ultrasound imaging method and the ultrasound imaging apparatus of an embodiment of the present invention, the exact distance between the catheter tip and the target location can be measured in real time, and the exact position of the catheter tip in the blood vessel can be dynamically determined in real time.

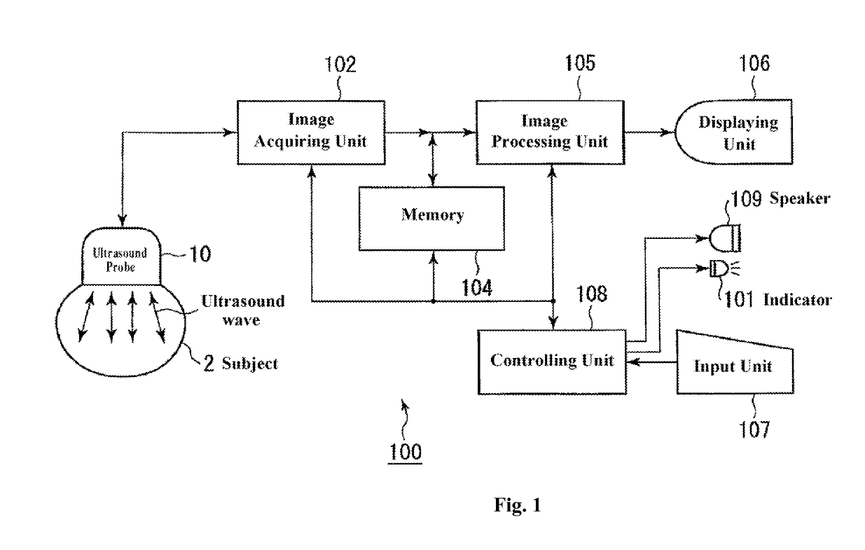

FIG. 1 illustrates a general structure of the ultrasound imaging apparatus 100 according to an embodiment of the present invention. As shown in FIG. 1, the ultrasound imaging apparatus 100 comprises an ultrasound probe 10, an image acquiring unit 102, a memory 104, an image processing unit 105, a displaying unit 106, an input unit 107, a speaker 109, an indicator 101, and a controlling unit 108.

The ultrasound probe 10 emits ultrasound waves to a region of interest in the body of a subject 2 (e.g., a patient) and receives the ultrasound echoes reflected from the region of interest in the body of the subject 2. The ultrasound probe 10 may include a probe array having piezoelectric devices arranged in an array therein, and perform the ultrasound sweeping of the region of interest including the target location and the target vessel into which the catheter is guided to be inserted, in a real time volume ultrasound scan mode. FIG. 3 illustrates a real time volume ultrasound scan mode. As shown in FIG. 3, the swept direction of the ultrasound probe 10 is substantially perpendicular to the longitudinal axis of the target vessel.

The image acquiring unit 102 acquires three-dimension scan data of the target location and the target vessel in the region of interest, and stores, in the memory 104, the acquired three-dimension scan data in the form of a three-dimension data matrix in ultrasound beam space, wherein the memory 104 is a mass storage such as a hard disk. In the process of performing the catheter insertion, the image acquiring unit 102 also acquires three-dimension scan data of the catheter tip. The size of the three-dimension matrix is Nₛₐₘₚₗₑₛ×N_{beams}×N_{frames}, where Nₛₐₘₚₗₑₛ is the number of samples in the depth direction, N_{beams} is the number of electrically controlled beams, and N_{frame} is the number of mechanically swept frames.

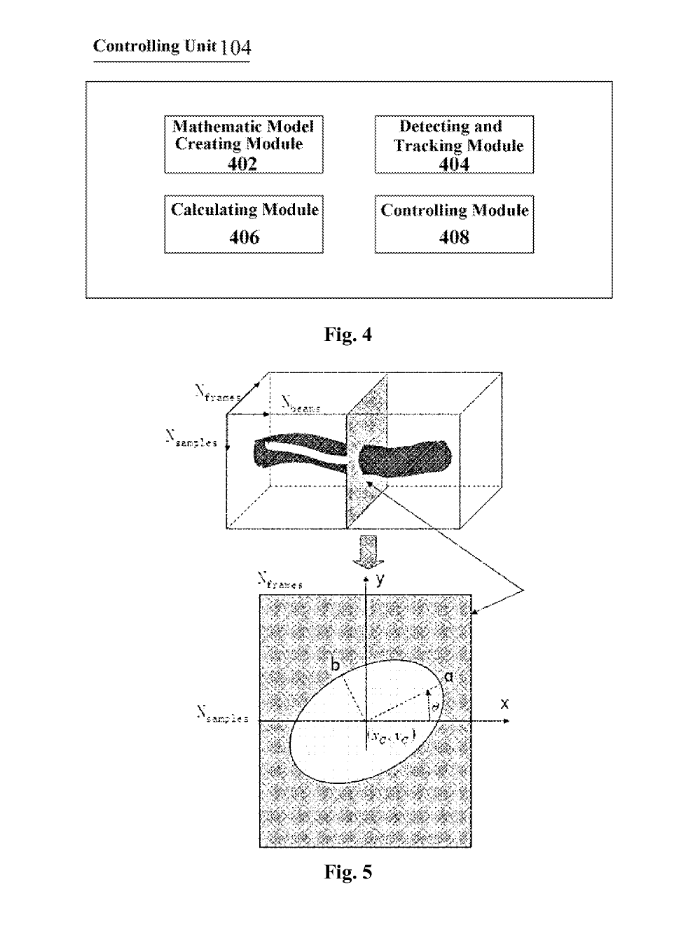

The controlling unit 108 calculates the centerline length of the target vessel for guiding the catheter insertion based on the acquired three-dimension scan data. FIG. 4 illustrates a general structural diagram of the controlling unit 108. As shown in FIG. 4, the controlling unit 108 comprises a mathematic model creating module 402, a detecting and tracking module 404, a calculating module 406 and a controlling module 408. Those skilled in the art will appreciate that the controlling unit 108 as shown in FIG. 4 may be implemented with software, hardware and/or firmware depending on actual needs. This implementation is readily achieved by those skilled in the art, and detailed description is no longer repeated for simplicity.

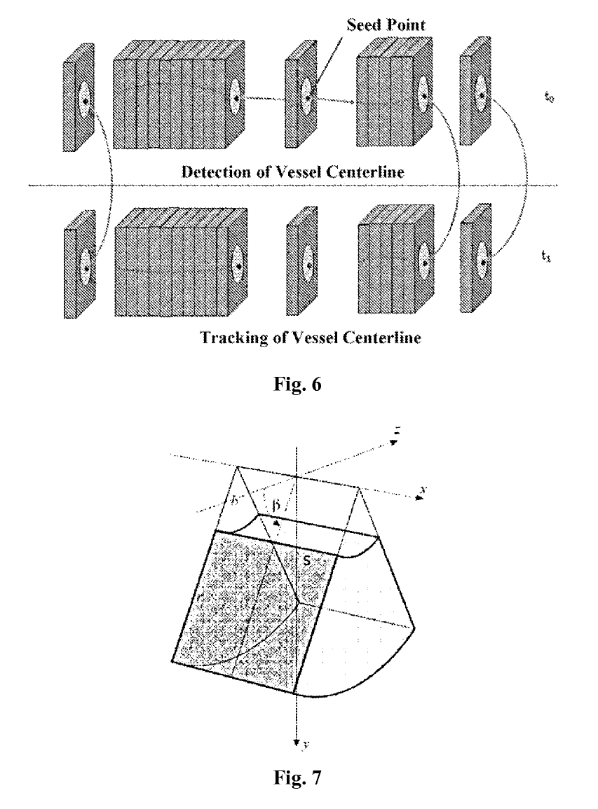

The mathematic model creating module 402 is used to create the mathematic model of the target vessel in three-dimension scan data. In an embodiment, the three-dimension scan data acquired by the image acquiring unit 102 include a plurality of two-dimension vessel frames along the longitudinal axis of the blood vessel, and thus the blood vessel in three-dimension beam space may be characterized by an ellipse queue, as shown in FIG. 6. Accordingly, the mathematic model creating module 402 uses parameters x_{c}, y_{c}, a, b and θ to define the ellipse cross-section of the blood vessel in each two-dimension vessel frame, as shown in equation (1) below:

$$\frac{{( x cos θ + y sin θ - {x}_{c} )}^{2}}{{a}^{2}} + \frac{{( y cos θ - x sin θ - {y}_{c} )}^{2}}{{b}^{2}} = 1 \qquad Equation ( 1 )$$

where, parameter x_{c} and y_{c} are centerline coordinates of the ellipse that respectively correspond to the horizontal component and vertical component of the coordinate plane where the vessel frame is located; parameters a and b respectively are the major axis and minor axis of vessel ellipse cross-section in the vessel frame; and parameter θ is the rotation angle of the major axis in the vessel ellipse cross-section with respect to x axis.

The detecting and tracking module 404 may detect and track the target vessel centerline in real time based on the three-dimension scan data by using the created mathematic model. FIG. 6 illustrates a method for detecting and tracking the vessel centerline in real time by the detecting and tracking module 404. In the method, the detecting and tracking module 404 detects and tracks the centerline in the vessel ellipse cross-section by applying the real time image segmentation algorithm. Specifically, the detecting and tracking module 404 may include a Kalman filter that employs two-dimension template matching, to detect and track the vessel centerline in real time on a frame-by-frame basis. As shown in FIG. 6, the detecting and tracking module 404 selects the initial center point in the acquired three-dimension scan data as the seed point, obtains the template of the vessel cross-section, sets centerline tracking parameters based on the seed point and the obtained template, initializes the Kalman filter and uses the Kalman filter to predict the next center point, then checks the validity of the prediction and performs template matching, and finally updates the Kalman filter based on the template matching in order to perform subsequent detecting and tracking. A method of detecting and tracking a vessel centerline in real time by using a Kalman filter is disclosed in U.S. patent application Ser. No. 12/645,781, entitled “Methods for automatic segmentation and temporal tracking” filed Dec. 23, 2009 by Patwardhan et al, the disclosure of which is incorporated herein by reference in its entirety. For each of input three-dimension ultrasound data, the detecting and tracking module 404 outputs the ellipse queue determined in accordance with equation (2) below,

I={(xᵢ,yᵢ,aᵢ,bᵢ,θᵢ)|i=1,2, . . . n} Equation (2).

The calculating module 406 automatically calculates the vessel centerline length based on the ellipse queue output by the detecting and tracking module 404. The vessel centerline length may be calculated in the Cartesian space that can describe the real ultrasound scan length. The centerline coordinates of the vessel cross-section in each two-dimension vessel frame are (n₁, n₂, n₃), where n₁ corresponds to x coordinate of the ellipse cross-section, n₂ corresponds to y coordinate of the ellipse cross-section, and n₃ corresponds to the number i of the two-dimension frame. The centerline coordinates of the vessel cross-section in each two-dimension vessel frame are converted from beam space to Cartesian space to calculate the exact length of the vessel centerline.

In an embodiment of the present invention, the transformation of the vessel centerline coordinates from three-dimension ultrasound beam space to Cartesian space comprises the following steps: in the first step, the target vessel centerline coordinates are transformed from three-dimension ultrasound beam space to three-dimension acquisition coordinate system; and in the second step, the target vessel centerline coordinates are transformed from three-dimension acquisition coordinate system to Cartesian coordinate system.

FIG. 7 illustrates a schematic diagram of a method for transforming the vessel centerline coordinates from beam space to Cartesian space. In an embodiment of the present invention, the three-dimension coordinate system employs a cylindrical coordinate system as shown in the figure. For integer values of the beam space coordinates (nᵢ, n₂, n₃) corresponding to the voxel location in beam space, the coordinates in the cylindrical acquisition system are given by equation (3) below,

r=n₁ n₁={1,2, . . . ,N₁}

s=shotangles(n₂) n₂={1,2, . . . ,N₂}

β=BImageAngles(n₃) n₃={1,2, . . . ,N₃} Equation (3)

where, the operator “shotangles” is a [1×N₂] vector of monotonously increasing azimuth offset for each beam measured in voxels, and “BImageAngles” is a [1×N₃] vector of monotonously increasing elevation angles in radians, wherein variables s and β do not necessarily have to be equidistance sample.

In an embodiment of the present invention, the transformation of coordinates from cylindrical coordinate system to Cartesian coordinate system is given by equation (4) below,

x=δs

y=δ(b+r)sin(β)

z=δ(b+r)cos(β) Equation (4)

where, δ is a constant which means space distance between two neighbor cube voxels, (x, y, z) are the final vessel centerline coordinates for calculating the vessel centerline length. After the vessel centerline coordinates are transformed from beam space to Cartesian space, the vessel centerline length is calculated in a space curve integral manner by equation (5) below,

$$L = {\int }_{L} d s \approx {\sum }_{i = 1}^{n} \sqrt{{( {x}_{i} - {x}_{i - 1} )}^{2} + {( y - {y}_{i - 1} )}^{2} + {( {z}_{i} - {z}_{i - 1} )}^{2}} \qquad Equation ( 5 )$$

where, n is the total number of sample vessel center voxels in Cartesian space.

In an embodiment of the present invention, the calculating module 406 comprises a coordinate transforming unit and a calculating unit. The coordinate transforming unit is configured to transform the vessel centerline from three-dimension ultrasound beam space to Cartesian space, wherein the coordinate transforming unit transforms the target vessel centerline coordinates from three-dimension ultrasound beam space to three-dimension acquisition coordinate system like cylindrical coordinate system, and transforms the target vessel centerline coordinates from three-dimension acquisition coordinate system to Cartesian coordinate system. The calculating unit is configured to calculate the target vessel centerline length based on the target vessel centerline coordinates in Cartesian space by equation (5).

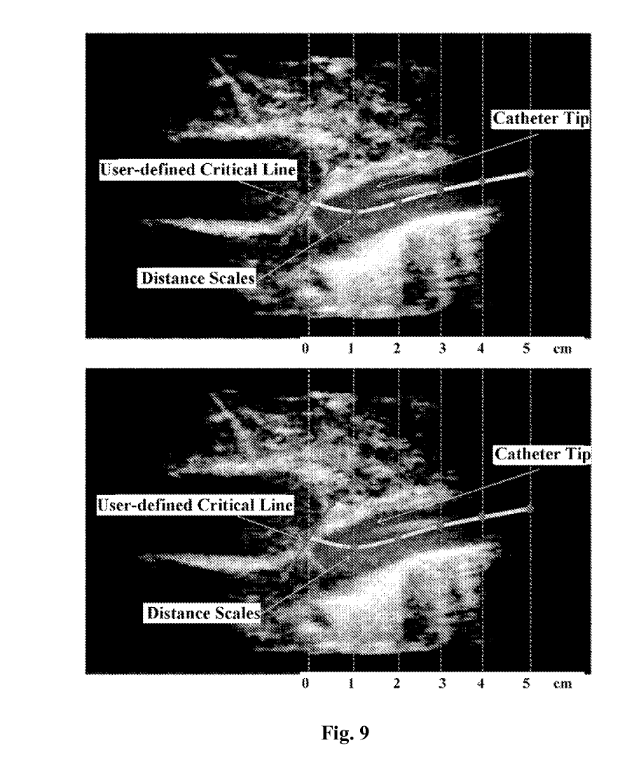

The image processing unit 105 is configured to mark distance scales from the target location based on the vessel centerline length calculated by the calculating module 406 in the controlling unit 108. The three-dimension image in Cartesian coordinate system is first projected onto two-dimension coordinate plane like XOY plane, thus obtaining a two-dimension ultrasound image and displaying it on the displaying unit 106, wherein the displaying unit 106 may include a CRT (Cathode Ray Tube) display or a LCD (Liquid Crystal Display) and the like. The obtained two-dimension ultrasound image may include the target location like a heart and the target vessel, when the catheter is inserted along the vessel centerline, the obtained ultrasound image may further include the ultrasound image of the catheter tip, and the displaying unit 106 is further configured to display the catheter tip in real time together with the target location and the target vessel centerline. A critical line may be defined at the target location (e.g., heart entrance) on the two-dimension ultrasound image, and the distance scale of the critical line is set to 0. Then, distance scales are automatically marked from the critical line located at the target location along the vessel centerline at predetermined intervals. The interval between two adjacent distance scales may be set in accordance with the accuracy required by a user, e.g., 0.5, 1.0, 1.5, 2.0 cm, etc., and the intervals may be the same or different. FIG. 9 illustrates a process of catheter insertion at two different time instants. As shown in FIG. 9, a critical line is set at the heart entrance, and distance scales marked from the critical line along the vessel centerline are 0, 1 cm, 2 cm, 3 cm, 4 cm, 5 cm, respectively, wherein there is the same interval between adjacent distance scales, i.e., 1 cm. In the upper figure of FIG. 9, the catheter is inserted along the vessel centerline and reaches near the distance scale less than 1.5 cm, while in the lower figure of FIG. 9, the catheter reaches near the distance scale more than 1.5 cm.

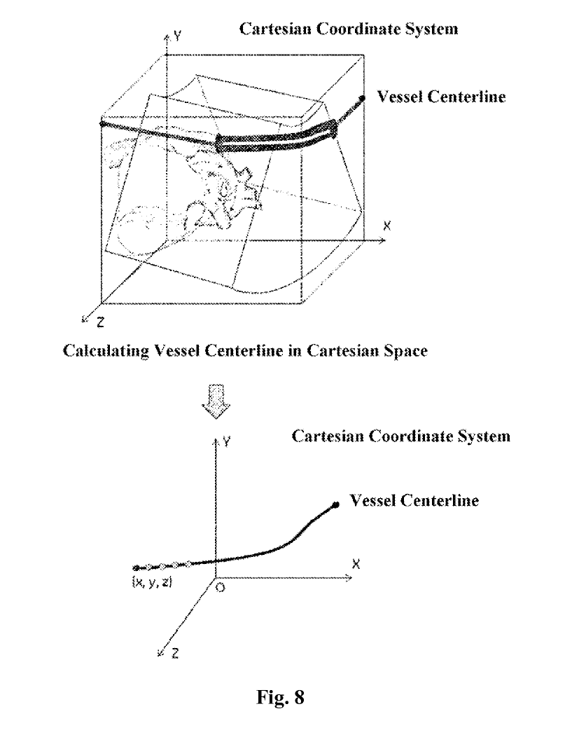

Alternatively, marking of distance scales described above may be performed on the three-dimension ultrasound image before the three-dimension ultrasound image is projected onto two-dimension coordinate plane, as shown in FIG. 8, and then the three-dimension ultrasound image marked with distance scales is projected onto two-dimension coordinate plane, in order to obtain the two-dimension ultrasound image marked with distance scale and display it on the displaying unit 106 for reviewing by a surgeon who performs the catheter inserting procedure.

The controlling unit 108 also comprises a controlling module 408 which performs various control functions for the ultrasound imaging apparatus. Alternatively, the ultrasound imaging apparatus 100 shown in FIG. 1 may further include a detecting unit (not shown) to determine in real time the position of the catheter tip in a blood vessel when the catheter having a position sensor disposed at its tip is used to perform the catheter inserting process. When it is determined that the catheter having a position sensor disposed at its tip is inserted into a predetermined distance from the critical line, the controlling module 408 may send a control signal to an alarm such as an indicator 101 and/or a speaker 109, to acoustically and optically alert the surgeon who performs the catheter inserting procedure, thereby informing the surgeon of the catheter being at or proximate to the predetermined position. The above-mentioned distance is determined by a surgeon based on the age, body shape of a patient, in conjunction with his past experience. For example, for premature infant, the predetermined distance may be set to about 1 cm from the critical line located at the heart entrance, and for full term infants, may be set to about 2 cm from the critical line located at the heart entrance.

A user may input the above-mentioned predetermined distance and distance scale interval and other operating data through input unit 107 such as keyboard, mouse, touch screen, and the input predetermined distance and distance scales may be stored in the memory 104. Furthermore, a user may operate the ultrasound imaging apparatus according to an embodiment of the present invention through the input unit 107, for example, control the shape and position of the image displaying on the displaying unit 106.

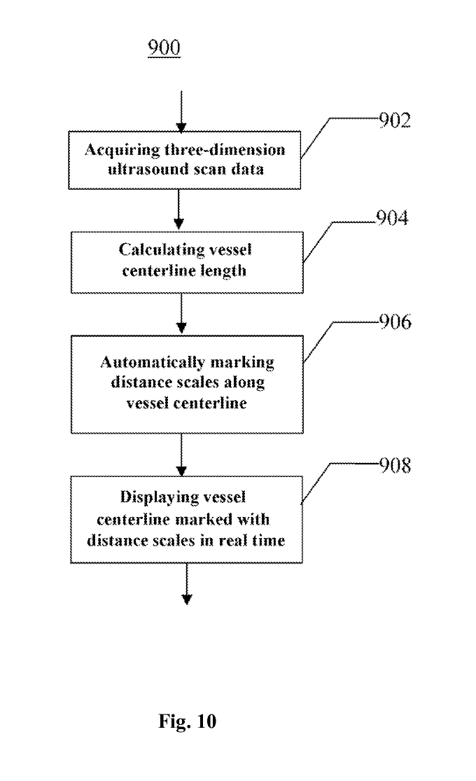

FIG. 10 illustrates a flowchart of an ultrasound imaging method according to an embodiment of the present invention. As shown in FIG. 10, In Step 902, three-dimension ultrasound scan data of the region of interest including the target location such as the heart and the target vessel into which the catheter is guided to be inserted and/or the catheter are acquired by using a real time volume ultrasound scan mode, and the acquired three-dimension scan data are stored in the mass storage in the form of three-dimension data matrix in ultrasound beam space for subsequent processing. When three-dimension scan data are acquired, the swept direction of the ultrasound probe is substantially perpendicular to the longitudinal axis of the target vessel, as shown in FIG. 2. The size of the three-dimension matrix is Nₛₐₘₚₗₑₛ×N_{beams}×N_{frames}, where Nₛₐₘₚₗₑₛ is the number of samples in the depth direction, N_{beams} is the number of electrically controlled beams, and N_{frame} is the number of mechanically swept frames.

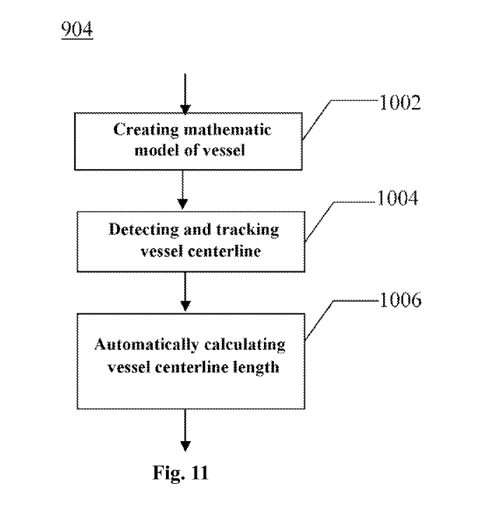

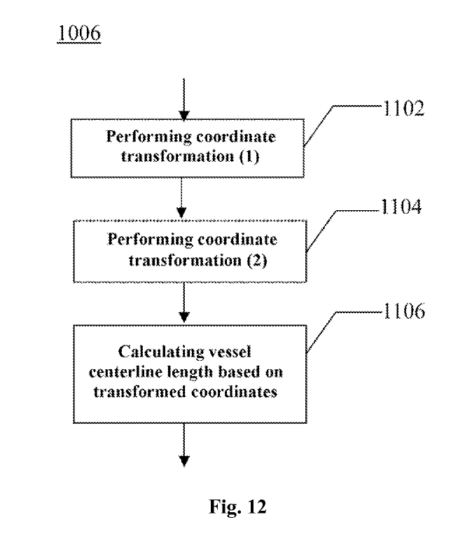

In Step 904, the centerline length of the target vessel is calculated based on the acquired three-dimension ultrasound scan data. FIG. 11 illustrates a method of calculating the target vessel centerline length based on three-dimension ultrasound scan data according to an embodiment of the present invention. As shown in FIG. 11, in Step 1002, the mathematic model of the target vessel in three-dimension ultrasound scan data is created. As described above, in an embodiment of the present invention, the acquired three-dimension ultrasound scan data include a plurality of two-dimension vessel frames of the longitudinal axis of the blood vessel, and therefore, the blood vessel in three-dimension beam space may be characterized by the ellipse queue shown in FIG. 6, in which the vessel ellipse cross-section in two-dimension vessel frame may be characterized by equation (1). In Step 1004, the target vessel centerline is detected and tracked in real time based on three-dimension ultrasound scan data by using the created mathematic model. In an embodiment of the present invention, the centerline of the vessel ellipse cross-section may be detected and tracked by applying a real time image segmentation algorithm. For example, the vessel centerline may be detected and tracked in real time on a frame-by-frame basis by using a Kalman filter. As shown in FIG. 6, the initial center point is selected in the acquired three-dimension scan data as the seed point, the template of the vessel cross-section is obtained, centerline tracking parameters are set based on the seed point and the obtained template, the Kalman filter that employs two-dimension template is initialized and the Kalman filter is used to predict the next center point, then the validity of the prediction is checked and template matching is performed, and finally the Kalman filter is updated based on the template matching in order to perform subsequent detecting and tracking. A method of detecting and tracking a vessel centerline in real time by using a Kalman filter is disclosed in U.S. patent application Ser. No. 12/645,781, entitled “Methods for automatic segmentation and temporal tracking” filed Dec. 23, 2009 by Patwardhan et al, the disclosure of which is incorporated herein by reference in its entirety. For each three-dimension ultrasound data, the ellipse queue determined by equation 2 may be obtained. Then in Step 1006, the target vessel centerline length is automatically calculated based on the detected and tracked target vessel centerline. FIG. 12 illustrates a method for automatically calculating its length based on the detected and tracked vessel centerline. In Step 1102, coordinate transformation (1) is performed to transform the vessel centerline coordinates (n₁, n₂, n₃) from three-dimension ultrasound beam space to three-dimension acquisition coordinate system, wherein n₁ corresponds to x coordinate of the ellipse cross-section, n₂ corresponds to y coordinate of the ellipse cross-section, and n₃ corresponds to the number i of the frame. In Step 1104, coordinate transformation (2) is performed to transform the vessel centerline from three-dimension acquisition coordinate system to Cartesian coordinate system. In an embodiment of the present invention, the three-dimension coordinate system employs a cylindrical coordinate system as shown in the FIG. 7. For integer values of the beam space coordinates (n₁, n₂, n₃) corresponding to the voxel location in beam space, the coordinates in the cylindrical acquisition system are given by equation (3). In an embodiment of the present invention, the transformation of coordinates from cylindrical coordinate system to Cartesian coordinate system is given by equation (4). Then in Step 1106, the vessel centerline length is calculated by using the vessel centerline coordinates in Cartesian space. In an embodiment of the present invention, after the vessel coordinates are transformed from beam space to Cartesian space, the vessel centerline length is calculated in a space curve integral manner by equation (5).

Returning now to FIG. 10, in Step 906, distance scales are automatically marked from the critical line located at the target location along the target vessel centerline based on the calculated vessel centerline length at predetermined intervals. The three-dimension image in Cartesian coordinate system is projected onto two-dimension coordinate plane like XOY plane, and the projected two-dimension ultrasound image is displayed in real time. The two-dimension ultrasound image comprises the target location such as the heart and the target vessel, and when the catheter is inserted along the vessel centerline, the catheter tip is also displayed in real time together with the target location and the target vessel. A critical line may be defined at the target location (e.g., heart entrance) on the two-dimension ultrasound image, and the distance scale of the critical line is set to 0. Then, distance scales are automatically marked from the critical line located at the target location along the vessel centerline at predetermined intervals. The interval between two adjacent distance scales may be set in accordance with the accuracy required by a user, e.g., 0.5, 1.0, 1.5, 2.0 cm, etc., and the intervals may be the same or different.

In Step 908, the target location and the target vessel centerline marked with distance scales are displayed on the display in real time. As shown in FIG. 9, a critical line is set at the heart entrance, and distance scales marked from the critical line along the vessel centerline are 0, 1 cm, 2 cm, 3 cm, 4 cm, 5 cm, respectively, wherein there is the same interval between adjacent distance scales, i.e., 1 cm. In the upper figure of FIG. 9, the catheter is inserted along the vessel centerline and reaches near the distance scale less than 1.5 cm, while in the lower figure of FIG. 9, the catheter reaches near the distance scale more than 1.5 cm.

Alternatively, marking of distance scales described above may be performed on the three-dimension ultrasound image before the three-dimension ultrasound image is projected onto two-dimension coordinate plane, as shown in FIG. 8. Then, the three-dimension ultrasound image marked with distance scales is projected onto two-dimension coordinate plane, in order to obtain the two-dimension ultrasound image marked with distance scale and display it on the displaying unit for reviewing by a surgeon who performs the catheter inserting procedure.

With the use of the ultrasound imaging apparatus and the ultrasound imaging method according to an embodiment of the present invention, since distance scales are marked along the vessel centerline, when the catheter is inserted along the blood vessel, it is possible to dynamically perform quantitative measurement of the distance between the catheter tip and the target location (e.g., critical line) in real time, and guide the catheter tip to right position. Furthermore, distance scales marked along the vessel centerline may dynamically be changed in real time with physiological events such as heart beats and/or catheter insertion, thus it is possible to exactly and directly guide the real time placement of the catheter tip, as are milestones when we are driving on road.

Although the present invention has been described with reference to specific embodiments thereof, it is not intended that the present invention be limited to those specific embodiments. Those skilled in the art will appreciate that various modifications, substitutions and changes may be made to the present invention. For example, one step or component in the above embodiments of the present invention may be implemented in a number of steps or components, or conversely, functions of a number of steps or components in the above embodiments of the present invention may be implemented in one step or component. However, these changes should fall within the protected scope of embodiments of the present invention without departing from the spirit of the present invention. In addition, some terms as used in this specification and claims of this application are to be considered as illustrative and not restrictive in character. Furthermore, depending on actual needs, all or some of features described in a specific embodiment may be combined in other embodiments of the present invention.

What is claimed is:

1. An ultrasound imaging method comprising steps of:
acquiring three-dimension scan data of a target location and a target vessel by using a real time volume ultrasound scan mode;
calculating a target vessel centerline length based on the acquired three-dimension scan data;
automatically marking distance scales from a critical line located at the target location along the target vessel centerline based on the calculated centerline length at predetermined intervals; and
displaying the target location and the target vessel centerline marked with distance scales in real time.

2. The method according to claim 1, wherein the step of displaying the target location and the target vessel centerline marked with distance scales in real time comprises:
projecting the three-dimension scan data marked with distance scales onto a two-dimension coordinate plane; and
displaying the projected two-dimension scan data in real time.

3. The method according to claim 1, wherein when the three-dimension scan data are acquired, the swept direction of the ultrasound probe is substantially perpendicular to the longitudinal axis of the target vessel.

4. The method according to claim 1, wherein the marked distance scales have the same or different intervals.

5. The method according to claim 1, wherein the target location is a heart, and the critical line is located at the heart entrance.

6. The method according to claim 1, wherein the step of calculating the target vessel centerline length based on the acquired three-dimension scan data comprises:
creating a mathematic model of the target vessel in the three-dimension scan data;
detecting and tracking the target vessel centerline in real time based on the three-dimension ultrasound scan data by using the created mathematic model; and
automatically calculating the target vessel centerline length based on the detected and tracked target vessel centerline.

7. The method according to claim 6, wherein the acquired three-dimension scan data include a plurality of two-dimension vessel frames along the longitudinal axis of the target vessel.

8. The method according to claim 7, wherein the step of detecting and tracking the target vessel centerline in real time based on the three-dimension scan data by using the created mathematic model comprises:
detecting and tracking the target vessel centerline by applying a real time image segmentation algorithm.

9. The method according to claim 8, wherein the step of detecting and tracking the target vessel centerline by applying the real time image segmentation comprises:
detecting and tracking the target vessel centerline in real time on a frame-by-frame basis by using a Kalman filter, in order to obtain the target vessel centerline coordinates of each two-dimension vessel frame in three-dimension ultrasound beam space.

10. The method according to claim 9, wherein the step of automatically calculating the target vessel centerline length based on the detected and tracked target vessel centerline comprises:
transforming the target vessel centerline coordinates from three-dimension ultrasound beam space to Cartesian space; and
calculating the target vessel centerline length by using the target vessel centerline coordinates in Cartesian space.

11. The method according to claim 10, wherein the step of transforming the target vessel centerline coordinates from three-dimension ultrasound beam space to Cartesian space comprises:
transforming the target vessel centerline coordinates from three-dimension ultrasound beam space to three-dimension acquisition coordinate system; and
transforming the target vessel centerline coordinates from three-dimension acquisition coordinate system to Cartesian coordinate system.

12. An ultrasonic imaging apparatus comprising:
an ultrasound probe configured to perform ultrasound sweeping of a region of interest including a target location and a target vessel in a real time volume ultrasound scan mode;
an imaging acquiring unit configured to acquire three-dimension scan data of the target location and the target vessel;
a controlling unit configured to calculate a target vessel centerline length based on the acquired three-dimension scan data;
an image processing unit configured to automatically mark distance scales from a critical line located at the target location along the target vessel centerline based on the calculated centerline length at predetermined intervals; and
a displaying unit configured to display the target location and the target vessel centerline marked with distance scales in real time.

13. The ultrasound imaging apparatus according to claim 12, wherein the image processing unit is further configured to project the three-dimension scan data marked with distance scale onto a two-dimension coordinate plane, and wherein the displaying unit is further configured to display the projected two-dimension scan data in real time.

14. The ultrasound imaging apparatus according to claim 12, wherein the swept direction of the ultrasound probe is substantially perpendicular to the longitudinal axis of the target vessel.

15. The ultrasound imaging apparatus according to claim 12, wherein the marked distance scales have the same or different intervals.

16. The ultrasound imaging apparatus according to claim 12, wherein the target location is a heart, and the critical line is located at the heart entrance.

17. The ultrasound imaging apparatus according to claim 12, wherein when a catheter is inserted along the target vessel centerline, the image acquiring unit is further configured to acquire the three-dimension scan data of the catheter tip, and the displaying unit is further configured to display in real time the catheter tip together with the target location and the target vessel centerline.

18. The ultrasound imaging apparatus according to claim 12, further comprising:
a detecting unit configured to determine the position of a catheter tip of the catheter having a position sensor disposed at its tip when it is inserted along the target vessel centerline.

19. The ultrasound imaging apparatus according to claim 18, further comprising:
an alarm configured to issue an alarm when the catheter tip provided with the position sensor has reached a predetermined distance from the critical line along the target vessel centerline.

20. The ultrasound imaging apparatus according to claim 19, wherein the alarm is a speaker or an indicator.

21. The ultrasound imaging apparatus according to claim 12, wherein the controlling unit comprises:
a mathematic model creating module configured to create the mathematic model of the target vessel in three-dimension scan data;
a detecting and tracking module configured to detect and track the target vessel centerline in real time based on the three-dimension scan data by using the created mathematic model; and
a calculating module configured to automatically calculate the target vessel centerline length based on the detected and tracked target vessel centerline.

22. The ultrasound imaging apparatus according to claim 21, wherein the three-dimension scan data acquired by the image acquiring unit include a plurality of two-dimension vessel frames along the longitudinal axis of the target vessel.

23. The ultrasound imaging apparatus according to claim 22, wherein the detecting and tracking module is configured to detect and track the target vessel centerline by applying a real time image segmentation algorithm.

24. The ultrasound imaging apparatus according to claim 23, wherein the detecting and tracking module further comprises:
a Kalman filter configured to detect and track the target vessel centerline in real time on a frame-by-frame basis, in order to obtain the target vessel centerline coordinates of each two-dimension vessel frame in three-dimension ultrasound beam space.

25. The ultrasound imaging apparatus according to claim 24, wherein the calculating module further comprises:
a coordinate transforming unit configured to transform the target vessel centerline coordinates from three-dimension ultrasound beam space to Cartesian space; and
a calculating unit configured to calculate the target vessel centerline length based on the target vessel centerline coordinates in Cartesian space.

26. The ultrasound imaging apparatus according to claim 25, wherein the coordinate transforming unit transforms the target vessel centerline coordinates from three-dimension ultrasound beam space to three-dimension acquisition coordinate system, and transforms the target vessel centerline coordinates from three-dimension acquisition coordinate system to Cartesian coordinate system.
